(19) United States
(12) Patent Application Publication
(10) Pub. No.: US 2011/0208112 A1
(43) Pub. Date: Aug. 25, 2011

(54) MEDICAL DEVICE FOR INJECTING LOCAL ANESTHETIC

(76) Inventor: Morteza Gharib, Hollidaysburg, PA (US)

(21) Appl. No.: 12/862,493

(22) Filed: Aug. 24, 2010

Related U.S. Application Data
(63) Continuation-in-part of application No. 12/189,661, filed on Aug. 11, 2008, now abandoned.

(51) Int. Cl. A61M 19/00; A61M 37/00

(52) U.S. Cl. 604/21; 604/512

(57) ABSTRACT

A medical device for injecting a liquid medicine into an organism. The device comprises a needle, an injection tube attached to the posterior end of the needle and a housing having a conduit at the distal end. The needle being placed inside the housing in such a way that the needle can move from a position where entire length of needle is situated within the housing and the conduit to a position where the needle is protruded out of the housing. The device allows an anesthesia provider to perform a nerve block more safely and efficiently. In one embodiment of the device, an injection pad is used to single handedly inject syringes of liquid medicine into the injection tube.

### CROSS-REFERENCE This application is continuation in part of utility patent application Ser. No. 12/189,661 filed on Aug. 11, 2008 titled “ MEDICAL DEVICE FOR INJECTING A LOCAL ANESTHETIC.”

### FIELD OF THE INVENTION

The invention relates generally to medical devices for injecting fluid solutions into an organism and more particularly to medical devices for injecting a local anesthetic.

### BACKGROUND

A nerve block is a procedure in which an anesthesia provider will place a regional anesthesia needle in very close proximity to a nerve within the human body and perhaps animals. A local anesthetic solution is injected through the needle and around the nerve in order to provide anesthesia and pain relief in an area of the body which is innervated by the targeted nerve.

In order to accomplish this procedure correctly, a majority of anesthesia providers use traditional nerve block needles which consist of an insulated steel shaft needle. At the rear end the needle is attached to an injection tube and a conductive wire.

A small battery-operated nerve stimulator device is attached to the rear end of the conductive wire. The anesthesia provider will advance the needle through the skin toward the targeted nerve. An assistant operates the nerve stimulator by sending decreasing amounts of electric current through the needle, causing a specific muscle contraction. This contraction indicates the proximity of the needle to that specific nerve. Then the assistant injects the local anesthetic solution into the injection tube. The solution goes through the tubing and the needle to be deposited near the desired nerve.

During the injection, the anesthesia provider needs to keep the needle in a constant direction and depth. This process is necessary in order to prevent nerve injury or accidental injection of the medicine inside a blood vessel or a nerve. Either one of which can have grave consequences.

It is known in the art that performance of a nerve block: (1) is a very delicate procedure, (2) can be time consuming, (3) takes a great amount of practice to master,(4) can result in accidental nerve injury or intravascular injection which can lead to grave consequences such as seizure, coma, and even death, (5) the needle can become dislodged as a result of the slightest hand movements, and (6) has the potential to cause needle injury to the anesthesia provider and medical assistants (7) it may also cause infection at the injection site if sterility precautions not maintained.

Today, more and more people are using ultrasound imaging devices to help them to visualize the nerve during the performance of a nerve block or alternatively both imaging device and nerve stimulator are used to locate the desired nerve. Using an ultrasound device to visualize underlying tissues, including nerves, the needle is advanced until needle tip is seen in close proximity of the target nerve and liquid medicine is injected.

There is a need in the field for a medical device for injecting a local anesthetic that reduces the risk of needle injury to medical staff, is easier to use than similar devices in the field today, provides for more needle stability than currently available needles and provides a protective cover for the needle to protect it from possible contamination from surrounding environment.

### SUMMARY

The present invention is directed to a device that satisfies these needs of reduction in the risk of negative outcomes associated with nerve block procedures, making the nerve block procedure easier to perform, and reduces risk of needle injury caused by regional anesthesia needles to medical staff. The device generally comprises a regional anesthesia needle mounted on the front end of a plunger, an injection tube attached to the rear end of the needle, an injection port attached to the rear end of the injection tube, a housing and a conduit. The device may further include a conductive wire attached to the rear end of the needle. If a nerve stimulator device is used, this wire can be connected to it to send electric signals from a nerve stimulator device to the needle tip. The housing is shaped with dimensions that allow the plunger to slide inside the housing with a certain amount of friction created between the plunger and the housing. The conduit is positioned at the front opening of the housing. The conduit guides the needle as the needle moves from a position where the needle tip is completely inside the conduit to a position where the needle is projecting out from the front of the conduit. In one variation of this embodiment the device is comprised of a housing with a longitudinal slot and the needle includes a radial arm placed within the longitudinal slot. The radial arm of the needle is used to advance the needle out of the housing and into the organism.

In an alternative embodiment, present invention further comprises an injection pad. The base of injection pad is coated with a layer of medical grade adhesives that allows the injection pad to be temporarily attached to the patient's skin or any other surfaces. The injection port at the rear end of the injection tube is attached to the top surface of the injection pad to provide stable injection port so that the anesthetic solution can be single-handedly directed into the injection tube. This injection pad may further include an electrically conductive knob placed through its thickness in such a way that the bottom portion of the knob touches the patient's skin. This knob on the top can be attached to the return wire of nerve stimulator device so the connection between the patient's body and the nerve stimulator device can be established.

In order to place a nerve block using a nerve stimulator device, the conductive wire of the needle is attached to the nerve stimulator.. The conduit front end is rested against the patient's skin over the targeted nerve. The anesthesia provider uses one hand to constantly hold the conduit against the patient's skin. The other hand is used alternatively to advance the needle into organism and also to operate the nerve stimulator. After the targeted nerve is located, the anesthesia provider has the option of using an assistant to inject the local anesthetic solution into the injection port, or as it was described in the above alternate embodiment, the provider can use the specifically designed injection pad to single handedly inject the local anesthetic into injection port. At the end of the procedure, the anesthesia provider will retract the needle completely inside the housing in such a way that needle tip will be positioned within the conduit and disposes of the needle safely. This will reduce the risk of needle injury to the anesthesia provider and other medical personnel. The present invention can be used along with imaging devices as well. While the conduit front end is rested against the patient's skin, the needle is advanced toward target nerve under direct vision of underlying body tissues.

These and any other features, aspects, and advantages of the present invention will become better understood with regard to the following description, appended claims, and accompanying drawings.

### DRAWINGS

FIG. 1 shows a prior art nerve block needle.

FIG. 2a shows a sectional side view of a version of the plunger for the invention.

FIG. 2b shows a sectional side view of a version of the housing for the invention.

FIG. 2 shows a sectional side view of a version of the invention.

FIGS. 3 and 3a show a sectional side view of a version of the invention.

FIG. 4a shows a front view of a version of the housing for the invention.

FIGS. 4b and 4c show front view of a version of the invention.

FIG. 4d shows a side view of a version of the invention.

FIGS. 5a and 5b show a sectional side view of a version of the invention.

FIG. 6 shows a sectional side view of a version of the plunger for the invention.

FIG. 7 shows an exploded sectional side view of a version of the plunger for the invention.

FIG. 8 shows a sectional side view of a version of the injection pad for the invention.

FIG. 9 shows a plan horizontal view of a version of the injection pad for the invention.

### DESCRIPTION

Referring to FIG. 1, a conventional nerve block needle is comprised of a hollow needle 30 that is connected to an injection tube 34 and a conductive wire 36. The injection tube 34 has an injection port 37 at its posterior end. The injection port 37 is used to introduce the local anesthetic into the injection tube 34. The posterior end of the conductive wire 36 may be connected to a battery operated nerve stimulator device. The conductive wire 36 enables pulses of electricity to be sent through the needle 30 in order to determine the distance between the needle 30 and the nerve that the anesthesia provider desires to block.

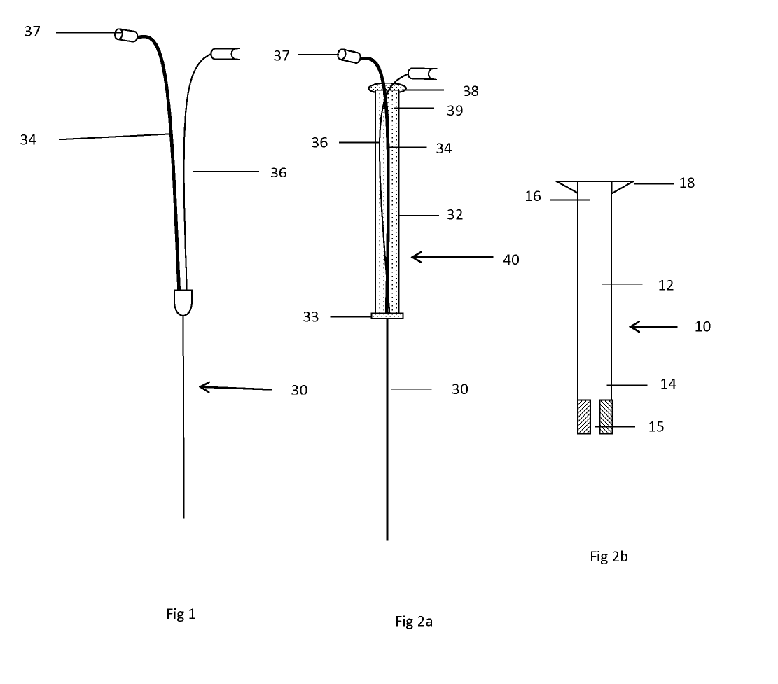

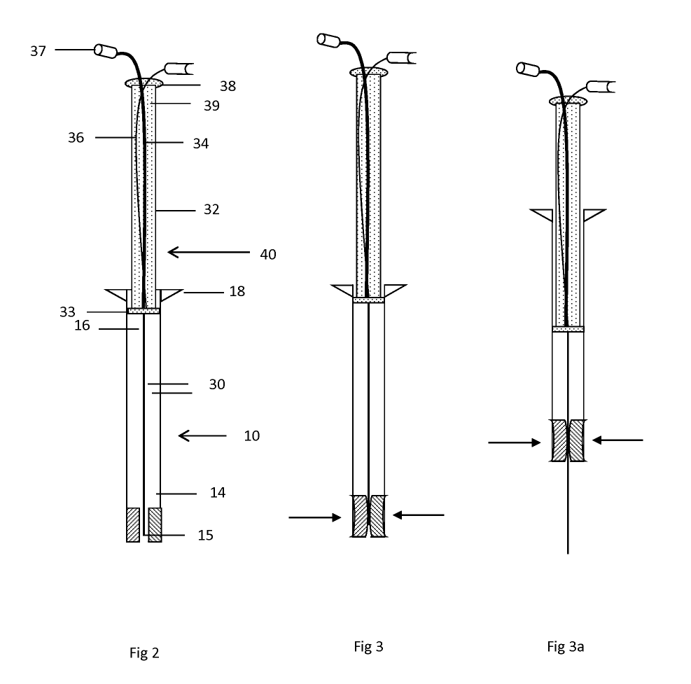

In the embodiment of the plunger 40 shown in FIG. 2a, the plunger 40 comprises a shaft 32 and an anterior ring 33. Plunger 40 may include a posterior knob 38 .The needle 30 is mounted to the front end of the plunger 40. Injection tube 34 is attached to the rear end of the needle 30, and injection port 37 is attached to the rear end of the injection tube 34. Injection port 37 can be used to inject liquid medicine through needle 30 and into organism. In certain embodiments, conductive wire 36 may be attached to the rear end of the needle 30 as well. If a nerve stimulator device is used, this wire can be used to send electric current into the needle 30. Injection tube 34 and wire 36 may be threaded through the shaft 32 and exit from the posterior end of the shaft 32 or they may pass through the anterior ring 33 and lay outside the plunger 40. The plunger 40 can be constructed out of any solid material, but is preferably made of plastic.

In the embodiment of the housing 10 shown in FIG. 2b, the housing 10 comprises a cylindrical body 12, an anterior end 14, and posterior end 16. The housing 10 may have finger flange 18 at its posterior end 16. The conduit 15 is attached to the anterior end 14 of the housing 10. The housing 10 can be any shape, but is preferably cylindrical. In addition, the housing 10 can be made of any solid material, but is preferably made of clear plastic to allow the user to see the needle 30 inside the housing 10. In certain alternative embodiments shown in FIGS. 3 and 3a, conduit 15 may be made out of compressible but resilient material so that it can be squeezed to create friction around needle 30 so that needle 30 can be kept at any given position relative to housing 10. Upon release of pressure the conduit maintain its straight shape and allow the needle to move freely again. This may be desirable to manipulate the needle 30 single handedly while holding the conduit against patient's body.

Referring to the embodiment of the invention shown in FIG. 2, the plunger 40 is inserted into the housing 10 and the needle 30 tip positioned within conduit 15. The internal diameter of the conduit 15 is made slightly larger than the outer diameter of the needle 30. This allows the needle 30 to pass through the conduit 15 in a guided fashion.

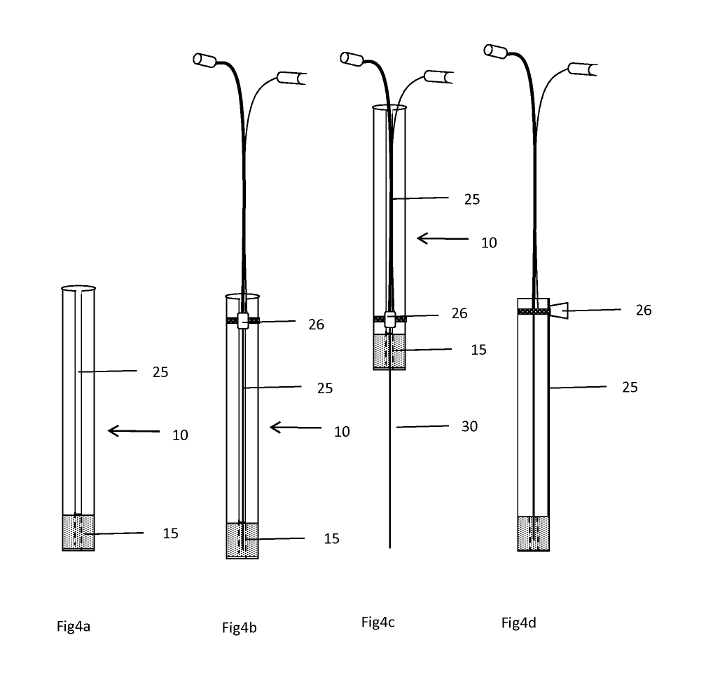

Referring to the embodiments of the invention shown in FIGS. 4a, 4b, 4c, and 4d, the housing 10 includes longitudinal slot 25 and the needle 30 includes side arm 26 positioned within slot 25. Conduit 15 is again attached to distal end of housing 10. Side arm 26 can be used to advance the needle 30 from a position where needle 30 tip positioned completely within conduit 15 to a position where needle 30 is projecting out of housing 10 and into the organism.

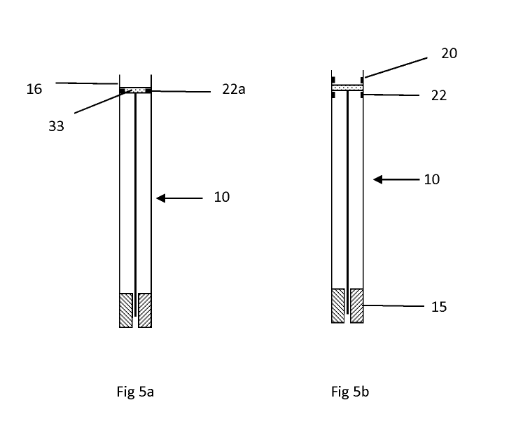

Referring to FIGS. 2, 3, and 4, it can be clearly seen that placing the needle 30 inside housing 10, and placing needle tip within conduit 15 provides a complete covering for entire length of the needle 30. The needle 30 can be protruded out of this covering and be retracted back inside the covering. This feature not only reduces risk of accidental needle injury to medical staff, it also protects needle 30 from possible contamination which may in fact reduces risk of infection at the injection site. Referring to FIGS. 5a and 5b, any embodiments of the current invention may further include means to releasably engage needle 30 with housing 10 in a position in such a way that needle 30 tip is situated within conduit 15 and not be touchable from outside. A slight force should be needed to release the needle 30 from this position. Such means may include anterior and posterior annular rings 20 and 22 formed on the interior surface of rear end 16, where ring 30 is maintained between these ring or including annular integral rib 22a formed on the interior surface of the rear end of the housing 10. The rib 22a is in snap fit engagement with ring 33. In an alternative way the rear end 16 of housing 10 can be made with smaller internal diameter compared to the rest of the housing 10 in such a way to create certain resistance to the movement of ring 33 within housing 10 rear end. Adding this means will maintain needle 30 tip inside conduit 15 at the beginning and the end of a nerve block procedure.

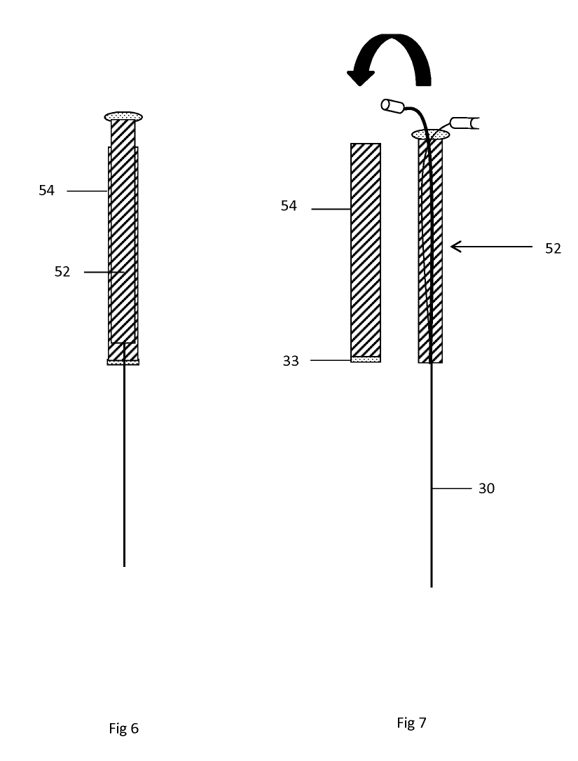

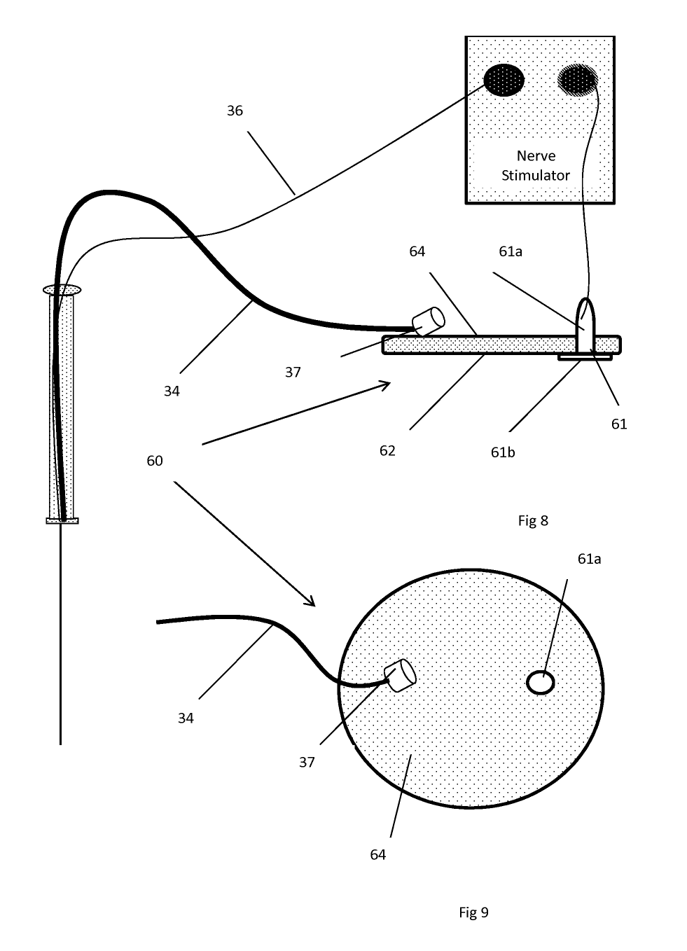

In an alternative embodiment of the invention shown in FIGS. 6 and 7, the invention comprises a fine tuning mechanism. As illustrated in FIGS. 6 and 7, the embodiment of the plunger 40 comprises two hollow cylinders 52, 54. The inner cylinder 52 is threaded on its outer surface, and the outer cylinder 54 is threaded on its inner surface. They function like a standard bolt and nut. The two cylinders 52, 54 move telescopically against each other by twisting both cylinders 52, 54 in opposite directions.. A user then rotates the inner cylinder 52 using the knob 38 that is attached to the inner cylinder 52. The rotation of the inner cylinder 52 causes the inner cylinder 52 to advance forward, away from the outer cylinder 54. Since the needle 30 is attached to the inner cylinder 52, the needle 30 advances in a forward direction. The fine tuning mechanism allows a user to achieve very fine movements of the needle 30 when the needle 30 is inserted into the organism.

An alternative embodiment of the invention comprises an injection pad. Embodiments of the injection pad are illustrated in FIGS. 8 and 9. The injection pad 60 can be made of any flexible material such as plastic or rubber. The injection pad 60 has a top surface 64 and a bottom surface 62. An injection port 37 is attached to the top surface 64 of the injection pad 60. The bottom surface 62 is covered with a layer of medical grade adhesives. This layer of adhesive may be covered with a removable backing. The injection pad 60 may further comprise conductive knob 61 placed through the thickness of the pad 60. Knob 61 has top portion 61a and flat bottom portion 61b. The Bottom portion 61b is adapted to contact to the patient's body. The bottom portion 61b may further be covered with layer of conductive gel. When injection pad 60 is positioned over a patient body, the flat bottom portion 61b of the knob 61 will touch the patient's skin. If a nerve stimulator device is used, then top portion 61a can be attached to the return wire of the nerve stimulator device. In this way connection between patient's body and the nerve stimulator will be established. The injection pad 60 provides a stable, immobile injection port 37, which allows the anesthesia provider to single-handedly attach, inject, and detach syringes of local anesthetics into the injection port 37. The injection pad 60 can be made in any size and shape, but is preferably made in the shape of a disc or square.

In any of embodiments described earlier marks may be placed over needle 30, plunger 40 or housing 10 to indicate the depth of penetration of needle 30 into an organism.

What is claimed is:

1- A method of injecting a liquid medicine into an organism, the method comprising the steps of;
a) Providing a housing, said housing having a conduit at the distal end,
b) Providing a hollow shaft needle, said needle comprising a tip and a proximal end, said needle being placed inside said housing and said needle tip positioned completely within said conduit,
c) Providing means for manipulating and advancing and retracting said needle in and out of said housing.
d) Placing said conduit against an organism and advancing said needle tip out of said conduit and into the organism in a desired position,
e) Injecting liquid medicine into said proximal end of said needle.

2- The method of claim 1, wherein said means comprises a plunger attached to the proximal end of said needle.

3- The method of claim 1, wherein said means comprises a radial arm attached to said needle and placed within a longitudinal slot made into said housing.

4- The method of claim 1, further including the step of retracting said needle back into said housing and positioning said needle tip completely inside said conduit.

5- The method of claim 1, further including the steps of;
a) Providing means for releasably engaging said needle with said housing in a position in such a way that said needle tip is maintained completely inside said conduit,
b) Applying certain amount of force to release said needle from said housing and manipulating and advancing said needle out of said housing and into an organism,
c) Retracting said needle back into said housing and engaging said needle with said housing using said means in such a way to position said needle tip completely inside said conduit.

6- The method of claim 1, further including the steps of;
a) Providing said needle with an injection tube and an injection port attached to proximal end of said injection tube,
b) Attaching syringes of liquid medicine to said injection port and injecting liquid medicine into said needle.

7- The method of claim 1, further including the steps of;
a) providing a conductive wire, said wire being attached to said proximal end of said needle,
b) Using said wire to send electric impulses to said tip of said needle.

8- The method of claim 1, further including the steps of;
a) Providing said housing with a conduit made of compressible but resilient material,
b) Manipulating and protruding desired length of said needle out of said housing,
c) Applying compressive pressure to said conduit and maintaining said needle in any given position relative to said housing,
d) Relieving pressure off said conduit and freeing said needle.

9- The method of claim 1, further including the steps of;
a) Providing said needle with an injection tube and an injection port attached to the proximal end of the injection tube,
b) Proving an injection pad, said pad comprising a bottom surface covered with a layer of adhesive and a top surface, said injection port of said needle being attached to said top surface,
c) Attaching said injection pad to a surface, using said injection port of said top surface to inject syringes of liquid medicine into said needle.

10- The method of claim 9, further including the steps of;
a) Providing said injection pad with a conductive knob, said knob comprising a top portion adapted to connect to a nerve stimulator device, and a flat bottom portion adapted to contact to a patient's body,
b) Attaching said injection pad to the patient's body and connecting a nerve stimulator device to said conductive knob.

11- A method of injecting a liquid medicine into an organism, said method comprising the steps of;
a) Providing a housing, said housing having a conduit at the distal end,
b) Providing a hollow shaft needle, said needle comprising a tip and a proximal end, said needle being placed inside said housing in such a way that said needle tip being placed completely inside said conduit,
c) Providing said needle with an injection tube and an injection port attached to the proximal end of said injection tube,
d) Providing first means for manipulating and advancing and retracting said needle in and out of said housing,
e) Providing second means for releasably engaging said needle with said housing in a position in such a way that said needle tip is maintained completely inside said conduit,
f) Placing said conduit against an organism, using first means to apply certain amount of force to disengage said needle from said housing and manipulating and advancing said needle out of said housing and into an organism,
g) Injecting liquid medicine into said proximal end of said needle,
h) Retracting said needle back into said housing and engaging said needle with said housing using said second means in such a way to position said needle tip completely inside said conduit.

12- The method of claim 11, further including the steps of;
a) providing a conductive wire, said wire being attached to said proximal end of said needle,
b) Using said wire to send electric impulses to said tip of said needle.

13- The method of claim 11, further including the steps of;
a) Providing said housing with a conduit made of flexible but resilient material,
b) Manipulating and protruding desired length of said needle out of said housing,
c) Applying compressive pressure to said conduit and maintaining said needle in any given position relative to said housing,
d) Relieving pressure off said conduit and freeing said needle.

14- A method of injecting a liquid medicine into an organism, said method comprising the steps of;
a) Providing a housing, said housing having a conduit at the distal end,
b) Providing a hollow shaft needle, said needle comprising a tip and a proximal end, said needle being placed inside said housing in such a way that said needle tip placed completely inside said conduit,
c) Providing said needle with an injection tube and an injection port attached to the proximal end of said injection tube,
d) Providing first means for manipulating and advancing and retracting said needle in and out of said housing,
e) Providing second means for releasably engaging said needle with said housing in a position in such a way that said needle tip is maintained completely inside said conduit,
f) Placing said conduit against an organism, releasing said needle from engagement with said housing and advancing said needle tip into the organism in a desired position and,
g) Injecting liquid medicine into said injection port and into said needle.

15- The method of claim 14, further including the steps of;
a) providing a conductive wire, said wire being attached to said proximal end of said needle,
b) Using said wire to send electric impulses to said tip of said needle.

16- The method of claim 14, further including step of providing said device with marks to indicate the depth of penetration of said needle in an organism.
